(19) United States
(12) Patent Application Publication
(10) Pub. No.: US 2010/0292762 A1
(43) Pub. Date: Nov. 18, 2010

(54) METHOD FOR CONTROLLING PHOTODYNAMIC THERAPY IRRADIATION AND RELATED INSTRUMENTATION

(76) Inventors: William J. Cottrell, Somerville, MA (US); Thomas H. Foster, Rochester, NY (US); Allan R. Oseroff, Buffalo, NY (US); Stephanie H. Pincus, Buffalo, NY (US); Tammy Lee, Rochester, NY (US)

Correspondence Address: BLANK ROME LLP, WATERGATE, 600 NEW HAMPSHIRE AVENUE, N.W., WASHINGTON, DC, 20037, US

(21) Appl. No.: 12/738,196

(22) PCT Filed: Oct. 20, 2008
(86) PCT No.: PCT/US08/80512

§ 371 Date: Jul. 22, 2010

Related U.S. Application Data
(60) Provisional application No. 60/980,918, filed on Oct. 18, 2007.

(51) Int. Cl. A61N 5/06

(52) U.S. Cl. 607/88

(57) ABSTRACT

In a first embodiment, there is no monitoring, and instead light is delivered according to a predetermined ‘recipe.’ In a second embodiment, the instrumentation provides a means for making the reflectance measurements during therapy without requiring the brief interruption. This device may therefore allow more accurate measurement of treatment-induced changes to the reflectance measurement. In a third embodiment, an adjustable aperture is used to constrict the area of a treatment beam.

### REFERENCE TO RELATED APPLICATION

The present application claims the benefit of U.S. Provisional Patent Application No. 60/980,918, filed Oct. 18, 2007. Related subject matter is disclosed in WO 2006/025940 A2, WO 2007/120678 A2, and PCT/US08/62494. The disclosures of the above-identified applications are hereby incorporated by reference in their entireties into the present disclosure.

### STATEMENT OF GOVERNMENT INTEREST

The work leading to the present application was supported by NIH Grants CA122093, HL66988 and CA55719. The government has certain rights in the invention.

### FIELD OF THE INVENTION

The present invention is directed to the monitoring of photodynamic therapy and more particularly to such monitoring using different types of light.

### DESCRIPTION OF RELATED ART

PCT/US08/62494 describes a method for delivering PDT using feedback control, wherein a dose metric(s) is monitored and the delivery of treatment light is tailored in response. However, the monitoring introduces an extra step.

1) Foster et al. (reference 1) is a 1996 paper which describes a two-irradiance delivery of 514 μm light used to treat mouse tumors. The two irradiances were 20 & 28 mW and 20 & 40 mW and the drug was Photofrin. No aspects of the therapy were monitored during the delivery. This reference anticipates a multiple (two)-irradiance PDT therapy but does not include human subjects.

2) Mitra and Foster (reference 2) is a 2004 paper which describes a change in light penetration depth (and subsequently fluence rate) in a mouse model. Changes to the fluence rate in the tumor result from changes to the light penetration depth, which in turn results from blood oxygenation changes and changes to tissue absorption. This reference anticipates changes to fluence rate in the treated tissue, but does not anticipate explicit changes to the irradiance at which PDT is being delivered.

3) Henderson, et al. (reference 3) is a 1992 paper which describes a well-known phenomenon called “self-shielding”, which is functionally very similar to prior art reference 2. Self-shielding involves absorption of light in the tumor tissue near the light source by the sensitizer, which reduces the fluence rate in underlying tissue. As the sensitizer bleaches and that region becomes less absorptive, the fluence rate in the underlying tissue increases. As in reference 2, this reference anticipates changes to fluence rate in the treated tissue, but does not anticipate explicit changes to the irradiance at which PDT is being delivered.

4) Foster et al. (reference 4) is a 1991 paper which describes a fractionated PDT delivery, wherein light is delivered at a first irradiance, then paused for some time, then delivered at that irradiance again. Treatment fractionation has become a well-known method for maintaining tissue oxygenation during PDT. This reference anticipates a multiple-irradiance therapy wherein one irradiance is zero. We do not have knowledge of any references which include fractionation with varying light intensities in the ‘light on’ step.

In another area, WO 2007/120678 A2 describes instrumentation for delivering PDT and making reflectance measurements. That instrumentation makes a brief interruption of treatment to make a reflectance measurement in the treatment area, which provides information on tissue optical properties, blood oxygen saturation, blood volume, concentration of photosensitizer, and other spectroscopy-accessible parameters. However, it would be desirable to eliminate the interruption.

To the best of the inventors' knowledge there is no prior art anticipating simultaneous therapy/reflectance monitoring. There are instances of monitoring fluorescence simultaneously with therapy, as is described in WO 2007/120678, and adjacently to therapy, also described in WO 2007/120678.

FIG. 1 shows the system disclosed in WO 2007/120678. As shown in FIG. 1, in the system 100, light from a fluorescence laser 102, a treatment laser 104, or a white light source 106 is selectively applied by a switch 108 under the control of a computer 110 through a treatment fiber 112 to a target lesion L and a perilesion margin P. Reflected or fluorescent light received from the lesion L and the perilesion margin P is received through detection fibers 114 and another switch 116 into spectrometers 118, which analyze the signals and supply them to the computer 110.

In yet another area, constricting the area of irradiation using an adjustable aperture, which maintains the irradiance, is well known in medical imaging using ionizing radiation. However, it is not known in the art to do so with a treatment field.

### SUMMARY OF THE INVENTION

It is an object of the invention to overcome the above-noted limitations of the prior art.

To achieve the above and other objects, in a first embodiment, there is no monitoring, and instead light is delivered according to a predetermined “recipe.”

In a second embodiment, the instrumentation provides a means for making the reflectance measurements during therapy without requiring the brief interruption as required by WO 2007/120678 A2. This device may therefore allow more accurate measurement of treatment-induced changes to the reflectance measurement.

In a third embodiment, an adjustable aperture is used to constrict the area of a treatment beam.

The embodiments can be used separately or combined with one another or with the techniques disclosed in the above-cited applications.

### BRIEF DESCRIPTION OF THE DRAWINGS

Preferred embodiments of the present invention will be set forth in detail with reference to the drawings, in which:

FIG. 1 is a schematic diagram showing a device disclosed in the above-cited patent applications, usable in at least one embodiment of the present invention;

FIGS. 2A and 2B are schematic diagrams showing a front end of a system according to at least one embodiment of the present invention;

FIGS. 3A-3F are plots showing relative spectra at different points in the system of FIGS. 2A and 2B; and

FIGS. 4A and 4B are schematic diagrams showing the use of an adjustable aperture to constrict the area of irradiation in at least on embodiment of the invention.

### DETAILED DESCRIPTION OF THE PREFERRED EMBODIMENTS

Preferred embodiments of the invention will be set forth in detail with reference to the drawings, in which like reference numerals refer to like elements throughout.

A first preferred embodiment provides a simpler delivery where there is no monitoring, and instead light is delivered according to a predetermined “recipe.” For example, this might unfold as:

1) Light delivered at 50 mW cm⁻², for 20 J cm ⁻²

2) Light delivered at 100 mW cm⁻² for the subsequent 80 J cm ⁻²

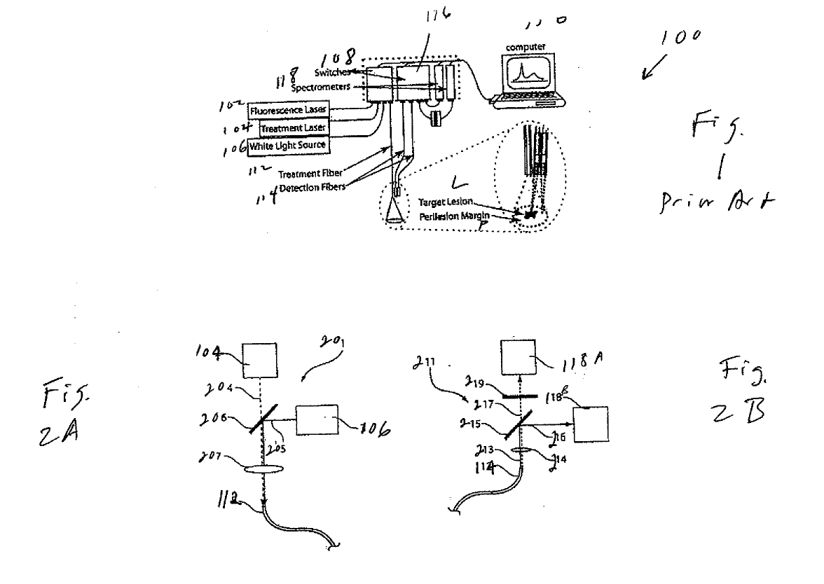

The specifics of the therapy can be determined empirically from results of clinical trials, which establish efficacies and pain thresholds as well as other relevant clinical results. The device of FIG. 1, or any other suitable device, can be used, in which case the computer can be programmed to deliver the light automatically according to the predetermined “recipe.”

In a second preferred embodiment, the instrumentation relates closely to the instrumentation and PDT system described in WO 2007/120678. The first preferred embodiment uses a front end that is usable with the system 100 described above.

FIG. 2A shows the front end 201. Treatment source 104 and reflectance source 106 generate treatment beam 204 and reflectance beam 205, respectively, Beams 204 and 205 are directed onto dichroic beam splitter 206, which combines the beams such that they are coincident. The beams are coupled into a treatment fiber 112 using coupling optics 207. The output of the treatment fiber is directed to a treatment region of the patient. This front end could be used directly with the PDT system of FIG. 1.

In an additional modification 211 to the system, shown in FIG. 2B, detection fiber 114 collects fluorescence and reflectance from the treatment region and directs it to the back end of the system.

Coupling optics 214 collimate the beam and direct it to dichroic filter 215 which splits the spectrum into a long wavelength region 217 and a short wavelength region 216. Long wavelength region 217 is directed through long-pass filter 219 to filter out the treatment beam before the region is measured by spectrometer 118A. Similarly, short wavelength region 216 is directed to spectrometer 118B. The short wavelength region of the spectrum contains reflectance information and the long wavelength region contains fluorescence information. Fluorescence and reflectance measurements can be made simultaneously using this instrumentation.

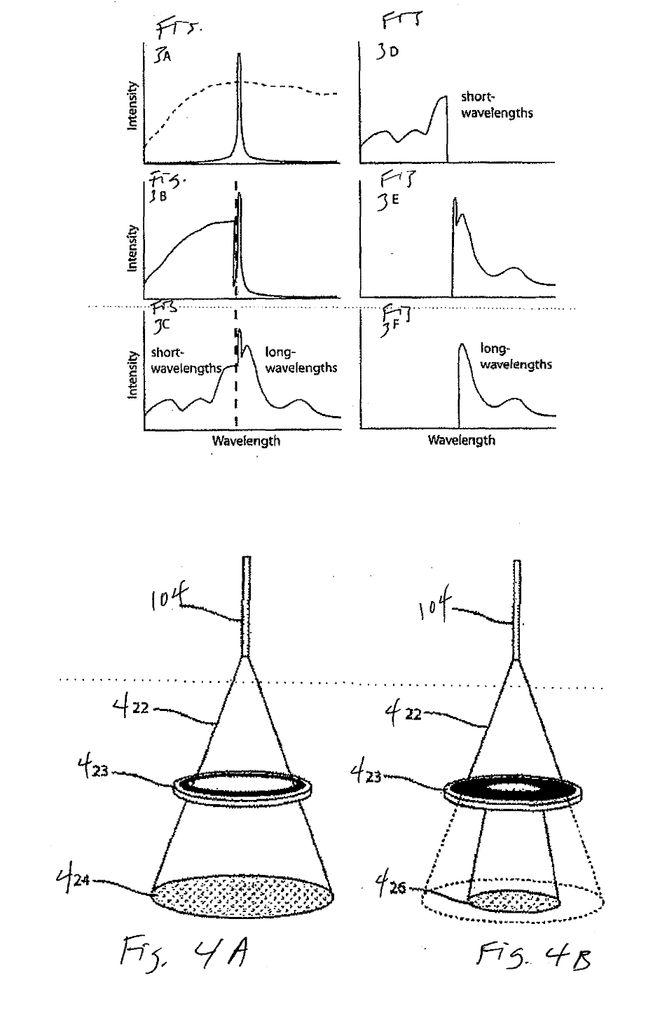

FIGS. 3A-3F show the relative spectra at different points in the system illustrating (3A) possible individual spectra from the treatment (solid) and reflectance (dashed) sources, (3B) combined spectra after the first dichroic filter, (3C) combined fluorescence and reflectance signals collected in the detection arm, (3D) content of short wavelength beam 216, (3E) long wavelength beam 217, and (3F) filtered long wavelength beam after second dicrhroic 219.

Alternate embodiments include:

1) A shutter or shutters which can be used to control delivery of treatment beam 204 and/or reflectance beam 205.

2) An optical filter between dichroic 215 and spectrometer 118B which filters out the treatment beam.

3) A 2×1 optical switch which collects light from multiple detection fibers and output that signal to back end 211.

4) Dissimilarly polarized treatment and reflectance beams, which are combined using a polarizing beam combiner instead of the dichroic filter.

5) An angled long pass filter 219 which directs the reflected treatment beam onto a detector (not shown).

A third preferred embodiment, providing adjustable constant-irradiance treatment field in PDT, will now be disclosed. This embodiment provides for an adjustable treatment field which maintains a constant irradiance at any size. As shown in FIGS. 4A and 4B, this embodiment includes a treatment beam source 104 which produces beam 422. In a first adjustment, beam 422 passes through adjustable aperture 423 in an open state to produce treatment area 424. In a second adjustment, beam 422 passes through aperture 423 in a partially closed state to produce reduced treatment area 426. Treatment area 424 and reduced treatment area 426 provide the same irradiance.

While preferred embodiments have been set forth above, those skilled in the art who have reviewed the present disclosure will readily appreciate that other embodiments can be realized within the scope of the invention. For example, embodiments disclosed separately can be combined. Also, numerical limitations are illustrative rather than limiting. Therefore, the present invention should be construed as limited only by the appended claims.

We claim:

1. A device for delivering photodynamic therapy, said device comprising:
a source of a first irradiance;
a source of a second irradiance;
optics for applying the first irradiance and the second irradiance to a target region; and
a controller for controlling the source of the first irradiance, the source of the second irradiance, and the optics for automatically providing the second irradiance to said target region in response to an application of the first irradiance to the target region.

2. The device of claim 1, wherein the controller controls the source of the first irradiance, the source of the second irradiance, and the optics in accordance to supply a predetermined power or energy to the target region.

3. The device of claim 1, wherein the source of the first irradiance and the source of the second irradiance are the same.

4. The device of claim 3, wherein the controller controls the source such that the second irradiance is at a higher intensity than the first irradiance.

5. A device for delivering photodynamic therapy, said device comprising:
sources for simultaneously generating a treatment beam and a reflectance beam;
optics for combining the treatment beam and the reflectance beam and for simultaneously directing said treatment beam and said reflectance beam onto a treatment region of a patient to produce a reflectance signal and fluorescence signal;
optics for separating said reflectance signal and said fluorescence signal;
a spectrometer for measuring said reflectance signal; and
a spectrometer for measuring said fluorescence signal.

6. A device for delivering photodynamic therapy, said device comprising:
a source and optics for delivering a treatment beam to a treatment region; and
a source and optics for delivering a reflectance beam simultaneously with said treatment beam to said treatment region; and
a spectrometer for monitoring reflectance spectra from said treatment region.

7. A device for delivering photodynamic therapy according to claim 6, further comprising a spectrometer for monitoring fluorescence spectra from said treatment region.

8. A device for delivering photodynamic therapy, said device comprising:
a source and optics for delivering a treatment beam to said treatment region; and
optics for adjusting the size of said treatment beam while maintaining the irradiance of said treatment beam.
